(19) United States
(12) Patent Application Publication
(10) Pub. No.: US 2003/0236474 A1
(43) Pub. Date: Dec. 25, 2003

(54) Seizure and movement monitoring

(76) Inventor: Balbir Singh, Spring, TX (US)

Correspondence Address: TROP PRUNER & HU, PC, 8554 KATY FREEWAY, SUITE 100, HOUSTON, TX, 77024, US

(21) Appl. No.: 10/178,587

(22) Filed: Jun. 24, 2002

(51) Int. Cl. A61B 5/00

(52) U.S. Cl. 600/595; 600/300

(57) ABSTRACT

An apparatus and method for monitoring movements of a patient is provided. If movements of the patient are detected to exceed predetermined thresholds, then a detector assembly determines that a seizure condition is present, and an alarm may be generated. In one arrangement, a sensor assembly includes one or more sound detectors to detect sound cause by patient movement. The detector assembly monitors the movement of the patient for determining if an abnormal movement or seizure condition is present. In another arrangement, an image sensor is used. A detector assembly monitors images produced by the image sensor to determine if the movements exceed predetermined thresholds. Other types of sensors can be used in other arrangements. The sensors can either be placed on a surface common to the patient or they can be attached to the patient.

### TECHNICAL FIELD

This invention relates to methods and apparatus to detect normal and abnormal body movements caused by conditions such as seizures, convulsions, and other movement disorders.

### BACKGROUND

To observe bio-electrical body functions of patients, electrodes may be attached to their bodies. For instance, electrical activity of the heart may be monitored by electrodes interconnected with the body. Unfortunately, electrodes are inconvenient and tend to become detached from the patient, with false alarms and patient anxiety being undesirable side effects. Furthermore, attached electrodes are likely to cause patient discomfort.

Conventional systems for monitoring patient movements are associated with various shortcomings. A need thus continues to exist for improved methods and apparatus for detecting seizures and other movements.

### SUMMARY

In general, according to one embodiment, an apparatus for detecting a movement disorder of a patient comprises a sensor assembly comprising one or more sound sensors, and a detector assembly adapted to receive an indication of sound detected by the one or more sound sensors. A detector assembly is adapted to determine if the movement disorder is present based on the indication.

In general, according to another embodiment, an apparatus for detecting a movement disorder of a patient includes a sensor assembly comprising one or more sensors selected from the group consisting of a sound sensor, a charge transfer sensor, an accelerometer, a micro-accelerometer, a seismometer, a geophone, a hydrophone, and a fiber-optic sensor. The sensor assembly is adapted to detect patient movement on a surface. A detector assembly adapted to receive an indication of the patient movement on the surface and to generate an alarm if the detector assembly determines a movement disorder is present.

In general, according to yet another embodiment, an apparatus includes an image sensor adapted to generate images of a patient, and a detector assembly coupled to the image sensor. The detector assembly includes an image-processing module adapted to process a series of images to determine if a patient seizure condition is present.

Other or alternative features will become apparent from the following description, from the drawings, and from the claims.

### BRIEF DESCRIPTION OF THE DRAWINGS

FIG. 1 is a simplified frontal perspective view of one embodiment of the present invention.

FIG. 2 is a simplified frontal perspective view of an alternative embodiment of the present invention.

FIG. 3 is an isolated frontal view of a portion of the embodiment depicted in FIG. 1.

FIG. 4 is a simplified schematic block diagram of the embodiment depicted in FIG. 1.

FIG. 5A depicts a waveform representing a patient's normal sleep activity recorded by an embodiment of the present invention.

FIG. 5B depicts a waveform representing a patient's 80-second seizure activity recorded by an embodiment of the present invention.

FIG. 5C depicts a waveform representing a patient's series of three 30-second seizures recorded over a three-hour period by an embodiment of the present invention.

FIG. 6 is a perspective view of another embodiment of the invention that uses sound detectors.

FIGS. 7 and 8 are block diagrams of different embodiments of a detector assembly.

FIG. 9 is a perspective view of a further embodiment of the invention.

FIG. 10 is a perspective view of a further embodiment of the invention that uses hydrophone detectors.

FIGS. 11A-11B illustrate waveforms generated by an example sensor.

FIG. 12 illustrates a detector assembly that can be attached to the body of a patient.

FIG. 13 shows an example arrangement of components in the detector assembly of FIG. 12.

FIG. 14 illustrates an embodiment of a position sensor used in the detector assembly of FIG. 13.

### DETAILED DESCRIPTION

In the following description, numerous details are set forth to provide an understanding of the present invention. However, it will be understood by those skilled in the art that the present invention may be practiced without these details and that numerous variations or modifications from the described embodiments may be possible.

Some embodiments of the present invention provide an apparatus for accurately monitoring a patient's body movements during periods of sleep. In particular, such embodiments monitor motor movements attributable to seizures and convulsions of patients having epilepsy or other seizure disorders, and motor movements attributable to periodic leg movements, tremors, respiration, mechanical cardiac functions, or any other motorics during periods of sleep. Generally, as used here, the term “seizure” also refers to any other type of movement disorder that a patient can experience.

As will be hereinafter described, detection of a seizure or other movement disorder is achieved without having to attach any detection apparatus to the patient. However, an embodiment in which a detection apparatus is attached to the body of the patient is not excluded from the scope of the present invention. Some embodiments measure patient movements essentially by relating mattress displacement to such motor movements. Mattress movements are detected using sensing devices placed on the mattress.

In other embodiments, other techniques for detecting movement of the patient are used. One alternative technique is to use audio detection. Another technique is to use video detection.

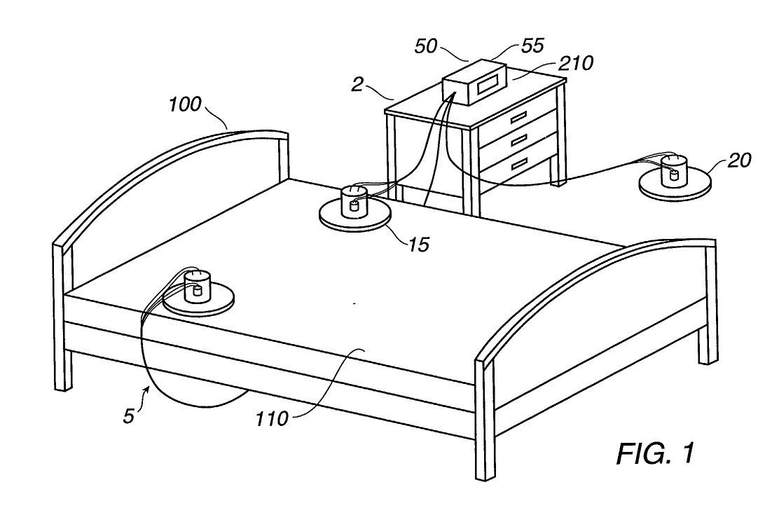

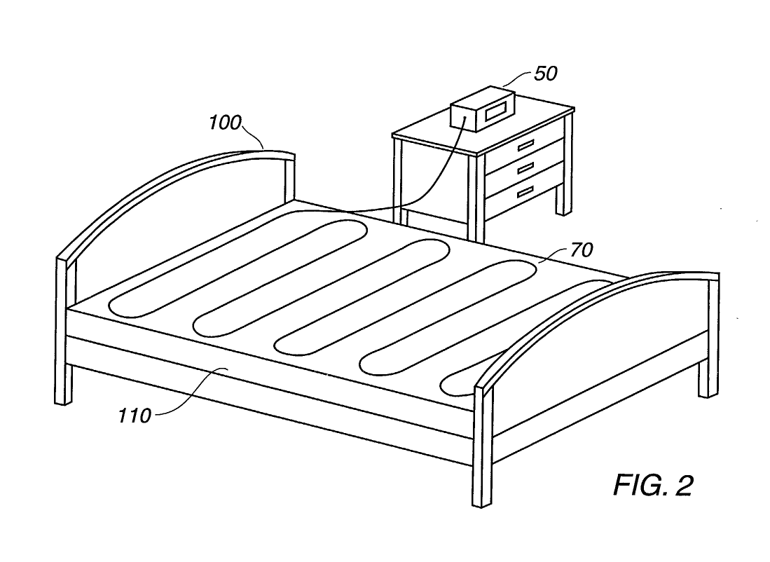

Referring to FIGS. 1 and 2, there are shown simplified perspective views of some embodiments of the present invention including a plurality of sensing devices 5 (e.g., geophone sensing devices, piezoelectric detectors, accelerometers, micro-accelerometers, seismometers, and so forth) disposed upon mattress 110 and adjacent or proximal to bed 100. In one embodiment, the plurality of geophone sensing devices are electrically interconnected by wires 5 with detector assembly 50. In the example arrangement of FIG. 1, geophones 10 and 15 of the plurality of geophones are disposed upon a mattress 110, with the geophone 10 and 15 placed adjacent or proximal a patient who may be lying on the mattress 110. More generally, the geophone or other type of sensing device is placed on a surface on which a patient is lying. However, to increase the sensitivity of the device, the sensing devices may be attached to the patient. The geophones can be connected to the detector assembly 50 with a wire or with wireless technology.

As patient body movements occur during sleep, corresponding displacements of mattress 110 occur. These displacements are communicated to at least one of geophones 10 and 15, which, in turn, communicate these signals to detector assembly 50 within its housing 55 as will be hereinafter described in detail.

Another geophone 20 is disposed proximal to the bed, e.g., on the floor, to establish a baseline or reference for signals that are attributable to environmental conditions, i.e., that are extraneous to the patient. Such environmental conditions may include vibrations from walking or from nearby elevators or escalators, vehicular traffic, etc. Typically, patient movements generate stronger signals in geophones 10 and 15, placed upon the mattress, compared to geophone 20, placed on the floor or on a structure away from the bed. The sensitivity of the plurality of geophones may be changed to obtain optimal results. In another arrangement, a plurality of sensors (instead of a single geophone 20) may be used to properly monitor vibrations attributable to environmental conditions extraneous to a patient's body movements.

Another embodiment of the present invention is depicted in FIG. 2, wherein instead of a plurality of geophones to sense a patient's motor movements during sleep, a plurality of fiber optics sensors 70 are used. Examples of a fiber optic sensor are described in U.S. Pat. No. 5,194,847. However, other types of fiber optic sensors can be used in other embodiments. The plurality of fiber optics sensors 70 are disposed in a sheet-like layer that may be conveniently and snugly placed immediately above the mattress cover disposed around mattress 110, or alternatively, disposed immediately beneath the top sheet.

In yet another embodiment of the invention, a hydrophone is used to detect movement of water caused by patient movement. The hydrophone is placed inside a water mattress or waterbed on which the patient is lying.

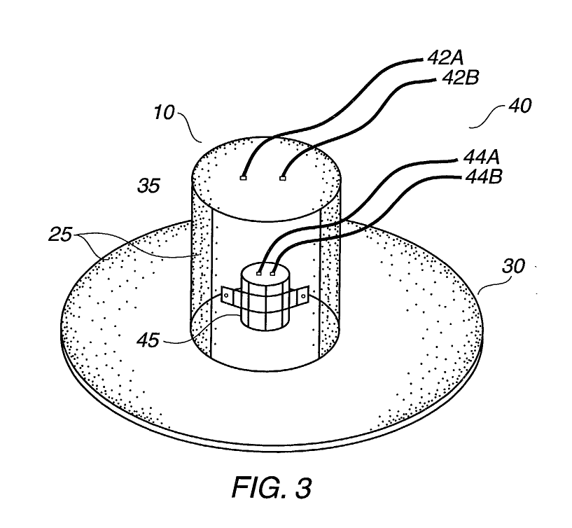

FIG. 3 shows a perspective view of the geophone 10, 15, or 20 that can be used by the embodiment of FIG. 1. One type of geophone includes a magnetic device that detects movements. Using a suspended magnet, the geophone, in response to movements occurring in its proximity, produces a proportional voltage through its winding. The amplitude of the output voltage is proportional to the intensity of the movement detected. Core-less DC motor member 45, akin to the motor incorporated into commonly used pager-vibrators, may be affixed atop the sensor's housing. This motor is turned on periodically to test the functionality of the geophone and detection system.

Thus, in FIG. 3, the geophone is shown having housing 35 mounted upon base plate 30. To avoid or reduce the likelihood of the geophone failing to properly detect signals, the vibrator motor member 45 is used to test proper operation of the geophone. By periodically activating vibrator member 45, a small movement is engendered in geophone. If the signal conditioning circuit 120 fails to receive a response from the geophone in response to the test, then a warning alarm or the like may be optionally generated to alert the operator that a geophone malfunction has occurred.

The geophone in one embodiment includes a cushion 25 in the surrounding housing 35 and base plate 30 to provide both electrical insulation and a physical barrier to prevent patient discomfort should inadvertent contact therewith occur.

The detector assembly 50 can be implemented in one of many different ways. For example, the detector assembly 50 can be implemented in a special-purpose system including hardware and software to perform detecting and processing. Alternatively, the detector assembly 50 can be implemented in a general-purpose personal computer in which appropriate software is located to perform the detecting and processing of signals from the sensing devices.

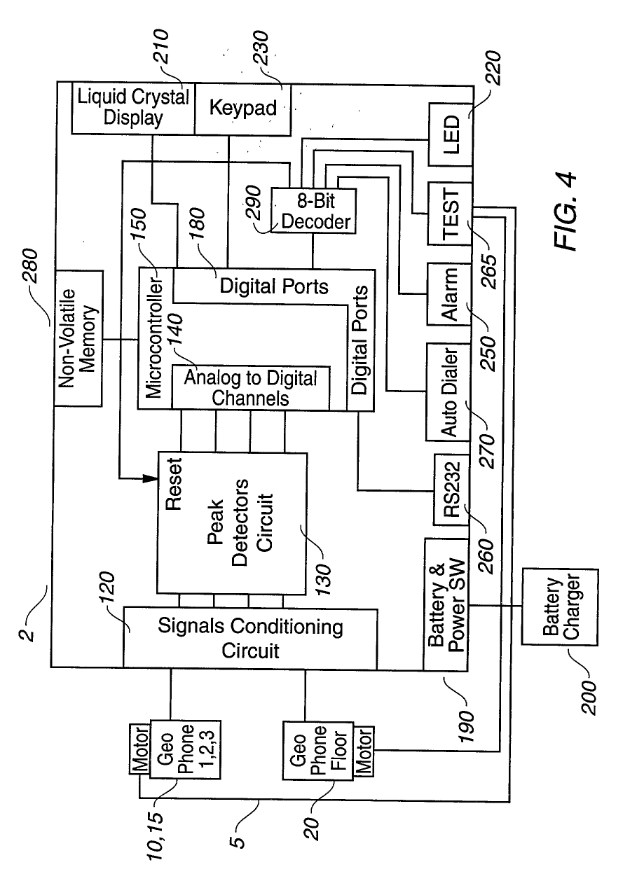

According to one embodiment, as shown in FIG. 4, the detector assembly 50 includes an LED light 220 (or some other visual indicator) to indicate if the system is operational. In one embodiment, the LED light blinks whenever the trigger threshold is exceeded. In FIG. 1, this condition corresponds to the amplitude of the waveform generated from either of geophones 10 and 15 disposed on the patient's bed being higher than the amplitude of the waveform generated from geophone 20 disposed on the floor. On the other hand, the LED light remains illuminated once an alarm condition has been detected, or in response to a malfunction. When a malfunction or a real alarm situation occurs, display (e.g., LCD) 210 optionally displays the nature of the malfunction or the alarm condition. Note that the arrangement shown in FIG. 4 is provided as an example only and is not intended to narrow the scope of the invention. Other embodiments can employ other arrangements.

To prevent accidental alarm-deactivation, the process of silencing an alarm in one embodiment requires sequential pressing of two keys. After an alarm is deactivated, the LED may optionally stay on until another, confirming sequence of keys is pressed. When a malfunction occurs, a simultaneous audio alarm and illumination of the LED light is provided.

As an additional safeguard against inadvertent shut off of an alarm, the turn-off keys may be programmed such that the sequential keys are not close to each other. A backup battery may also be provided for uninterrupted monitoring of patients' sleep even if power failure occurs. Circuit protection may also be provided to prevent any 115V current from being communicated to a geophone situated on the patient's bed, which may pose a safety hazard.

The detector assembly 50 can be programmed to pick up sustained seizures lasting for more than a preset length of time and a preset number of short but frequent seizures. The various settings, which may vary according to a patient's needs and specific type of seizure or other movement disorder, are programmable in software, ROM (read-only memory), or the like. Thus, the duration of a patient's seizure and the frequency thereof prerequisite to triggering an alarm condition depends upon the patient's particular needs.

When a seizure condition is detected, than an alarm is generated. The alarm can be an audio or visual alarm generated in the detector assembly, or it can be an external alarm. A remote alarm can also be activated to notify family members or caregivers. The remote alarm is located at some remote location, such as a nurse station, the home of a family member, 911 service, etc.

The remote alarm can be transmitted across telephone lines, wireless links (e.g., a cellular system), a data network (e.g., the Internet), and other communications channels. The detector assembly 50 includes an interface to communicate over one of these communications channels.

The detector assembly 50 can also include a storage medium to store a pre-recorded message. Once the detector assembly 50 has established a connection with a remote entity, the pre-recorded message can be played. The receiving party acknowledges the message by activating a predetermined sequence of keys (such as keys on a telephone or keyboard).

The detector assembly 50 is also able to store multiple numbers to call. The numbers can be stored in some predetermined priority order. If a first call is not acknowledged, the next number is called. This is continued until all numbers have been exhausted or one of the calls is acknowledged.

In another embodiment, a cardiac telemetry system or a similar but custom-made centralized telemetry unit is used to receive raw data from the sensors. The received data is displayed by the cardiac telemetry system on a screen (instead of an EKG trace). Seizure detection software can be loaded into the cardiac telemetry system to analyze the waveform produced by the received data. If a seizure condition is determined based on the analyzed waveform, the seizure detection software causes an alarm to be generated. The cardiac telemetry system can be located remotely from the patient being monitored. For example, the patient can be located at home while the telemetry system is located at a medical clinic, doctor's office, a hospital, or a central monitoring station.

As hereinbefore described, the plurality of geophones or the like that detect patient movements may be either placed on a patient's bed or disposed on the bed side-rail, affixed to the head-board or foot-board or even attached to the patient.

By comparing the cumulative analog signals received by plurality of geophones 10 and 15 disposed upon mattress 110 or alternatively received by various other types of motion sensors, or a combination thereof, and the base line signal received by geophone 20 disposed upon the floor or the like, the incidences of motor movements engendered by a patient may be continuously monitored by conditioning circuit 120, as shown in FIG. 4.

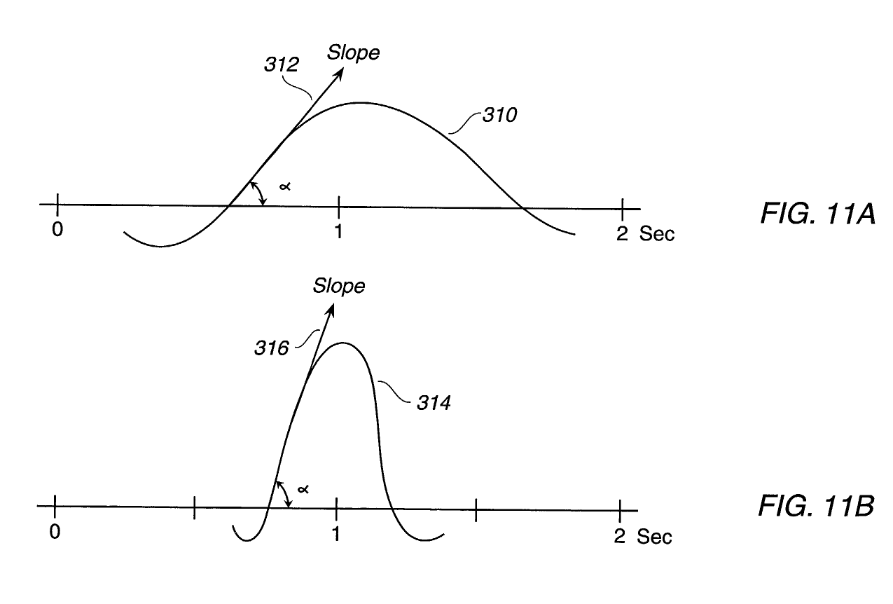

The conditioning circuit 120 uses filters and other components to amplify or attenuate the waveform incoming from the plurality of geophones 10 and 15 to a sufficient amplitude that may be input to the peak detectors circuit 130 that counts the peaks periodically (e.g., every second). In one example, the geophones' signals are terminated and then amplified to 0-2.5V full-scale signals. The conditioned signals are calibrated such that the voltage conditioned from each geophone is equal to the same intensity of the movement of each geophone. A peak detecting circuit 130 is used to measure the highest voltage generated at each geophone. This detection is periodically performed (such as every second) to measure the highest intensity of the movement every interval (e.g., second). According to one embodiment, this peak is reset by software in the detector assembly periodically (e.g., every second). A low-pass filter is included in peak detecting circuit 130 to filter any power noise (e.g., 50-60 Hz noise) from the input signals. Conditioning circuit can also select or reject a wave based on the characteristics of the waveform including angle of the slope (FIGS. 11A, B)

The peak voltages that are periodically detected are passed through an analog-to-digital converter 140. The analog signals are converted to digital signals representing the peak intensity that is proportional to the highest movement intensity during the periodic interval. A microcontroller or microprocessor 150 is used to perform a plurality of tasks as will be hereinafter described. Upon power up, the microcontroller/microprocessor 150 executes a conventional start-up sequence. In particular, the microcontroller/microprocessor 150 resets all the circuitry depicted in FIG. 4, and fetches the firmware from its non-volatile memory. It next interfaces with the user through keypad 230 and display 210 to set the intervals, movement intensity threshold, number of movement episodes to constitute an alarm condition, number of repeated movements in sequence to trigger alarm 250, and to set the operating mode to monitor, idle, and setup modes.

The microcontroller/microprocessor 150 coordinates the determination of whether detected movements are due to extrinsic causes. By comparing the signal level of geophones 10 or 15 with the reference level from floor geophone 20, this determination is readily made. If only the floor movement is detected then, the signal generated is deemed to be extraneous and is consequently ignored. Suitable software or the like enables the three peak detected signals to be read from the plurality of geophones disposed on the bed. The peak with the highest intensity is compared with the preset threshold value. If the movement is above the set intensity, the LED 220 is caused to blink, thereby indicating that a patient's movement has been detected.

Detected patient movements are recorded in a non-volatile memory 280 for some period of time (e.g., twelve hours or longer). The collected data can be downloaded to a personal computer via an RS232 or another type of port 260. The microcontroller/microprocessor 150 communicates externally through input/output digital ports 180. Numeral 290 represents an 8-bit addressable latch 1-of-8 decoder. Due to the limited number of digital I/O lines on microcontroller/microprocessor 150, latch decoder means 290 is used to read a specific address code from the I/O port which corresponds to an address of an output device such as auto dialer 270, alarm 250, LED 220, and test motor 265. An auto dialer 270 includes a contact switch activating an external auto dialer device.

A keypad 230 is provided for setting the time interval and period related to patient seizure detection. By making a suitable keypad-based request to the operational software, the recorded time of a patient's motor movement may be displayed. The display 210 is provided to display pertinent alphanumeric information indicative of the status of the patient's sleep behavior. According to one embodiment, Start/Stop switches may also be provided via a programmed set of two numeric keys to start or stop monitoring a patient's sleep activity.

The detector assembly 50 is powered via conventional battery charger adapter 200. The adapter 200 preferably of one example includes a lead-acid battery charger suited to battery 190, and regulates and charges a battery 190 from a 120-VAC power source. The battery 190 is used to provide a source of DC power for operation without external power source.

A toggle switch connected to the combination of adapter 200 and battery 190 provides a convenient way to switch off the svstem. In case power should fail, the battery assures continuous, uninterrupted operation of the detector assembly 50.

If a patient is detected to have experienced a motor movement within an interval, then the time for such movement is recorded in nonvolatile memory. The memory has the capacity to store up to some predetermined time period (e.g., twelve hours or longer) of data in one-second intervals. Alternatively it can be made to store all the raw waveform for a specified time period or complete data of all the seizures or just the length, intensity and the time of the seizures. Recorded raw waveform can be downloaded and analyzed. To increase the accuracy of the device, this information can be used to reprogram the device about the slope, intensity and other characteristics of the waveform of a particular patient. If the patient's motor movement continues and exceeds the programmed value, the microprocessor/microprocessor activates external and visual alarm 250.

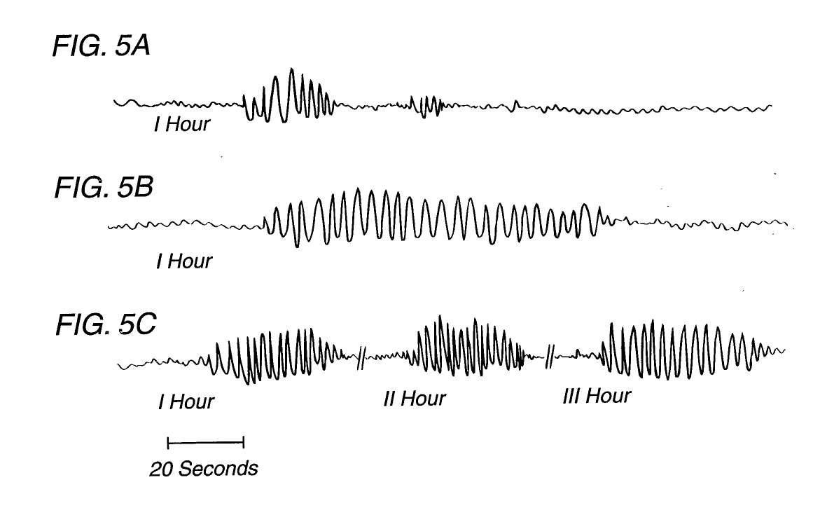

Referring now collectively to FIGS. 5A, B, and C, there is depicted representative waveforms, collected by the plurality of sensing devices. FIG. 5A shows a waveform of a patient's normal sleep activity. On the other hand, FIG. 5B shows the waveform corresponding to a patient having a seizure approximately 80 seconds in duration. In response to the waveform, an alarm indicating that a seizure is occurring is triggered in the detector assembly 50. Similarly, FIG. 5C shows an illustrative representation of three small seizures of about 30 seconds duration each, spread over a three-hour period.

The internal computer instructions used to implement the software in the detector assembly 50 may be stored in a storage medium and executed to accept keyboard input indicating whether an incidence of seizure intervals in a particular period should trigger an alarm. Thus, the peak detectors in the detector assembly 50 are driven by the underlying software or the like to detect and measure the peaks. Then, the microcontroller/microprocessor 150 assesses whether a particular series of waveforms are above the amplitude and length threshold; if such waveforms are below the threshold, then no seizure condition is considered to have occurred.

It is another feature and advantage of some embodiments of the present invention that the warning alarms and associated display may be communicated either locally or remotely to medical practitioners, healthcare personnel, or family. If the patient does not deactivate an alarm in the detector assembly 50, as will happen if the patient is, indeed, having a seizure, then the microcontroller/microprocessor 150 activates a remote alarm in another part of the house or in a nursing station or the like. This alarm may be connected to the detector assembly 50 with a wire or may include a wireless remote alarm controlled with electromagnetic signals or the like. For situations in which no one resides in the same house as a particular patient, the microcontroller/microprocessor of the present invention may be programmed to activate auto-dialer 270 to dial a predetermined telephone number and to play a prerecorded message. In one example, the telephone number summons a monitoring station or may summon a family member or “911.” A monitoring station may be able to interact with the microcontroller/microprocessor to deactivate the alarm and also change the monitoring settings, if needed. Instead of a telephone line, other communications media can be used, such as radio signals, wireless circuits of a cellular system, the Internet, or any other media can be used to transmit an alarm condition to a family member or a monitoring station.

In another embodiment, the geophones may be placed on a patient's bed along with a plurality of flexible strips of plastic or any other material that can be spread under the bed sheet and connected to each other. Thus, a geophone may be placed atop this arrangement to enhance its sensing function. Alternatively, instead of such strips, suitable wires and the like may be used. As another alternative embodiment, a liquid or air-filled mattress may be used to enhance the sensitivity of sensors to vibrations caused by a patients motor movements during seizures, convulsions, or other movement disorders. In this embodiment, one spot on one of the corners of the mattress may be made of low resistance material. A geophone may then be placed on this spot to pick up even the smallest vibrations created in the liquid or air.

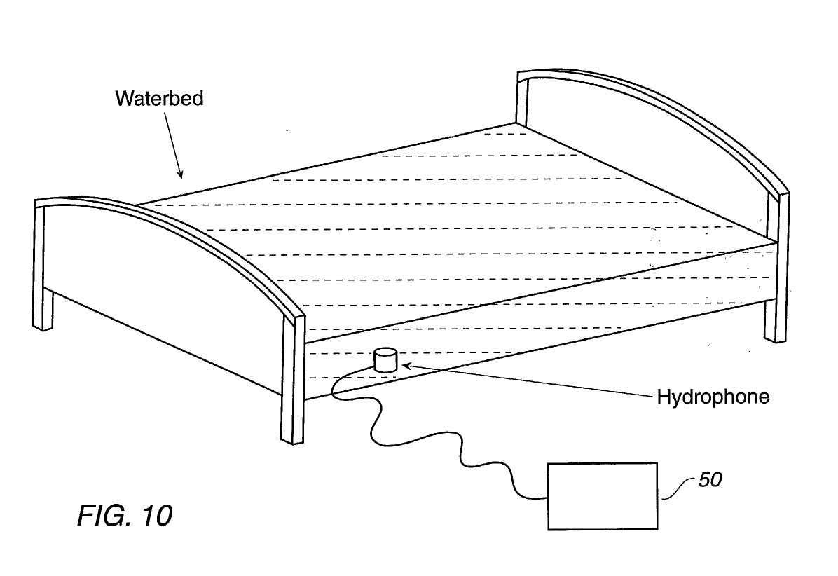

In other embodiments, other suitable patient-movement sensing devices based upon piezoelectrics, charge-transfer sensor, hydrophone, fiber optics, microwaves, infrared, and ultrasound may be used in addition to or instead of geophones. Also, another type of sensing device that can be used is an accelerometer or a micro-accelerometer. Yet another type of device is a seismometer, which is based on the molecular electronic transfer principle. One example seismometer that may be used includes a seismometer made by PMD Scientific Inc., based in Bloomfield, Conn. As hereinbefore described, such sensors may either be positioned upon a patient's bed or attached to a patient (to enhance sensitivity). In the case of microwave, infrared, and ultrasound-based motion sensors, they may be situated on an adjacent wall or a ceiling above the bed. In case of a hydrophone, as shown in FIG. 10, the hydrophone is placed inside the waterbed or a water-filled mattress.

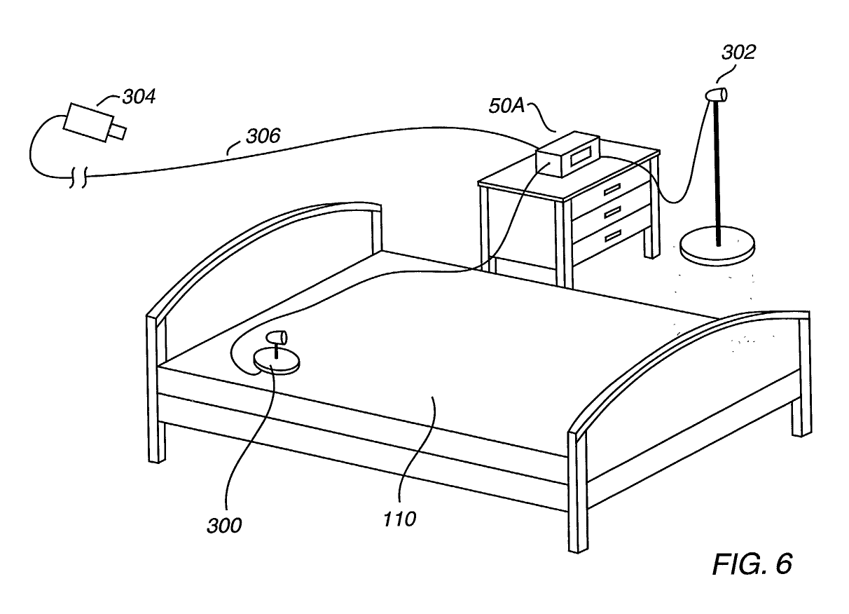

As shown in FIG. 6, in accordance with another embodiment, sensing devices each including sound detectors are used for determining a seizure condition of a patient. In the example arrangement of FIG. 6, a first sensing device 300 that includes a sound detector is placed on the mattress 110 (or other surface on which a patient is situated), and another sensing device 302 that includes a sound detector is positioned in the proximity of the bed for detecting environmental noise to provide a reference. Both sound detectors 300 and 302 are electrically connected to a detector assembly 50A, which is similar to detector assembly 50 except that it includes components to process sound signals. In one example, the sound detectors 300 and 302 are microphones. The microphones detect noise and sound made by movement of the patient, which are provided to the detector assembly 50 to diagnose movement and seizure conditions. The sound detector 300 can be attached to the mattress 110 (as shown) or to another bed structure. Also, the sound detector 300 can be attached to a blanket, a quilt, or the like, which can be made of a special material to enhance conduction and generation of sound to detect movement-induced noise. Although only one sound detector 300 is illustrated as being placed in the proximity of the patient, other embodiments can use multiple sound detectors 300.

The detector assembly 50A analyzes the audio data from the sound detectors 300 and 302 to determine if noise is coming from the bed or is due to external or environmental noise. In case of noise from the bed, the detector assembly 50A determines if the noise exceeds thresholds set for a normal range, which may indicate an abnormal movement or a seizure condition.

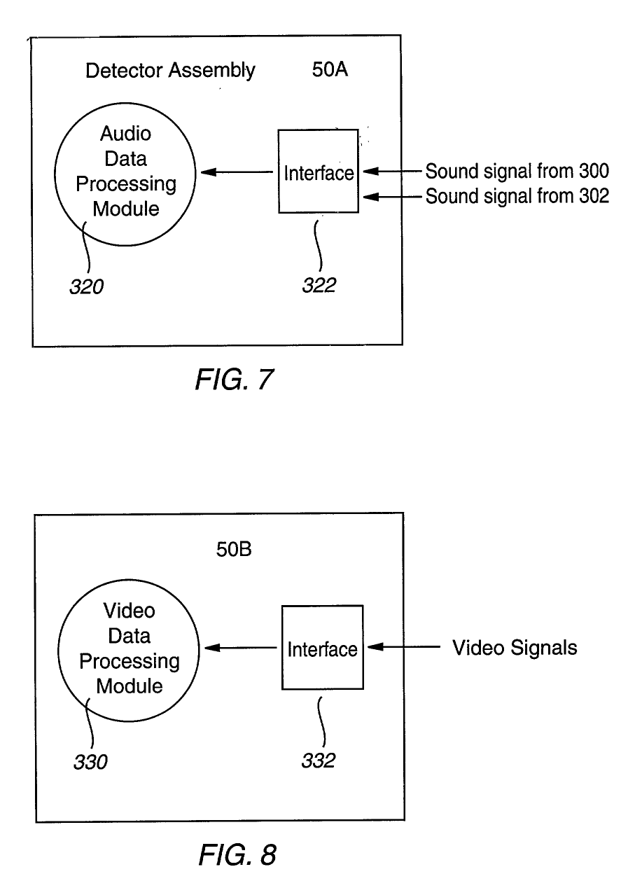

As shown in FIG. 7, an audio data processing module 320 (implemented as software or a combination of software and hardware) is provided in the detector assembly 50A. The audio data processing module 320 detects for a predetermined characteristic (e.g., a steep slope of the signal waves and/or rapid succession of peaks of sound for some predetermined period of time) that represents violent movement of the mattress 110. Sound signals from the sound detectors 300 and 302 are received by an interface 322, which includes appropriate analog-to-digital circuitry and other circuitry. The digitized sound signals are provided to the audio data processing module 320 for processing.

For example, as shown in FIGS. 11A and 11B, two waveforms 310 and 314 representing detected sound due to patient movement are shown. The first waveform 310 has a first slope 312. The second waveform 314 has a second slope 316 that is steeper than the first slope 312. The steeper slope is an indication of more violent movement of the patients. The slope is measured by an angle α. The larger the value of α, the steeper the angle. The microprocessor can be instructed to count waves with specific characteristics of the waveform, including an angle of slope and voltage. Similar waveforms can be generated using other types of sensors, which can be similarly processed by the detector assembly 50.

Optionally, a video camera 304 is provided in the room in which the patient is located. The video camera 304 records the patient's movement continuously. The patient's movement (or lack thereof) over some predetermined time period (e.g., a few minutes) can be recorded and previous data erased automatically and continuously.

The video camera 304 is electrically connected by a cable 306 or wireless technology to the detector assembly 50A. If the detector assembly 50A determines an abnormal condition, the detector assembly 50A sends an indication to the video camera 304. In response to this indication, the video camera 304 saves the segment of video data that pertains to the patient's movement during the period of the abnormal condition. It can be programmed to save a brief segment before and after the seizure as well. This saved segment can later be reviewed to determine what had happened. Although not shown, the video camera 304 may also be connected to some recording device, such as a digital storage device or a videocassette recorder, to save the video data associated with abnormal conditions.

In another embodiment, instead of using the video camera 304 to only record movement of the patient, a detector assembly 50B as shown in FIG. 8 can receive video data from the video camera 304. In this case, the video camera 304 is used as a sensor. A video signal is provided to an interface 332, which decodes the signals into images provided to a video data processing module 330. A sequence of digitized images of the patient is analyzed by the video data processing module 330 to determine if the patient is moving more than expected. The video data processing module at 330 compares one image to the next to determine their differences, and based on their differences, patient movement. A seizure condition is characterized by a series of rapid patient movements for some predetermined period of time. If the detector assembly 50B determines that the movements exceed preset limits, an alarm is generated.

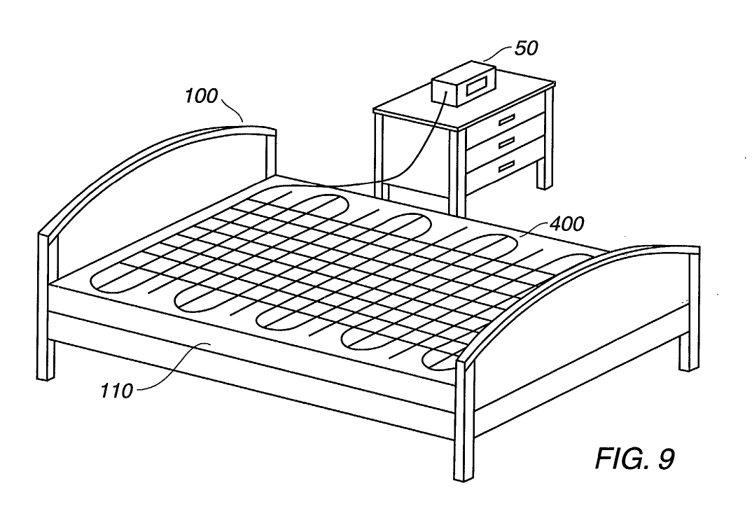

Referring to FIG. 9, in an alternative embodiment, another type of sensor device is used. In this case, sensors 400 that are based on the charge transfer (QT) principle are used in combination with a conducting material (such as a wire mesh or a thin metal foil) spread under the patient. The sensors can be used to detect all movements of the patient. One example of charge transfer sensors that can be used include the QProx ™ sensor sold by Quantum Research Group, Ltd., based in Pittsburgh, Pa.

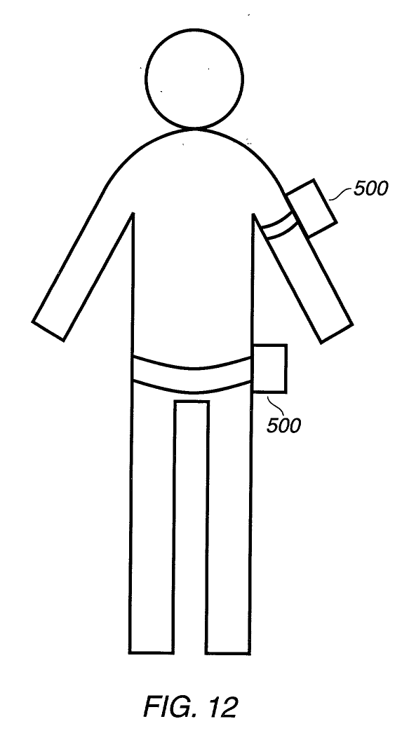

In alternative embodiments, a detector assembly for detecting seizure condition of a patient can be worn on the body of the patient. For example, as shown in FIG. 12, a detector assembly 500 can be attached to the body of a patient. As illustrated, the detector assembly 500 can be attached to the waist of the patient (such as on a belt) or to a limb (e.g., arm, leg, etc.) of the patient. The detector assembly 500 is configured to detect two conditions of the patient: movement of the patient and inclination of the patient. The detector assembly 500 detects for violent movement of the patient, such as those characterized by a seizure condition. However violent movement alone does not necessarily indicate that the patient is experiencing a seizure. To confirm that the patient is experiencing seizure, the detector assembly 500 also detects if the patient is no longer vertical (the inclination of the patient). A patient experiencing seizure may fall down, in which case the detector assembly 500 will detect that patient is now horizontal instead of vertical. The combination of violent movements and the patient no longer being in a vertical position is an indication that the patient may be experiencing a seizure condition. In response to detection of this combination, the detector assembly 500 generates an alarm.

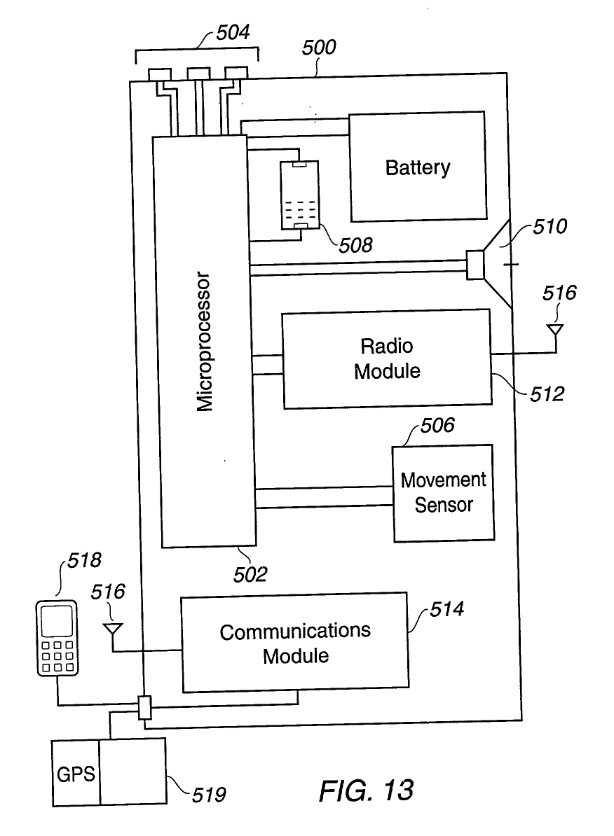

Alternatively the detector assembly 500 can be instructed to generate an alarm in response to detecting just sustained violent movement or whenever a horizontal position is detected even without a seizure. The alarm can be an audible alarm produced by the detector assembly 500. Alternatively, or in addition to the audible alarm, the detector assembly 500 includes a communications module 514 (FIG. 13) to send wireless signals to a base station over an antenna 516. The communications module 514 in the detector assembly 500 can also be connected to a wireless telephone to perform the communication. The base station can automatically dial preprogrammed numbers of family members, a central monitoring station or 911, to provide a notification of the abnormal condition. The device can be instructed to analyze the slope of the seizure waveform as well. When used away from home, the detector assembly can be connected to a cell phone 518 to activate an autodialer to alert family or place a call to 911. In remote locations without cell phone systems, a GPS/satellite two-way pager module 519 can be connected to the detector assembly 500. This module 519 can radio the location, alarm condition, and identity of the patient to a central monitoring station.

FIG. 13 shows an example arrangement of components in the detector assembly 500. The detector assembly 500 includes a microprocessor or microcontroller 502. The microprocessor 502 is connected to a keypad 504 to allow a user to provide input to the microprocessor 502. The keypad 504 may allow the user to turn off the detector assembly 500 or to provide other settings. The microprocessor 502 is also connected to a movement sensor 506. In one embodiment, the movement sensor 506 includes an accelerometer device. Alternatively, the movement sensor 506 includes a micro-accelerometer, geophone device, a piezoelectric device, and so forth. The movement sensor 506 is adapted to detect movement of the detector assembly 500. Signals representing the magnitude and the frequency of the movement are provided by the movement sensor 506 to the microprocessor 502, which processes the signals and analyses the characteristics of the waveform including the angle of the slope, to determine whether the detector assembly 500 is experiencing movement that is characterized by seizure condition of a patient.

The microprocessor 502 is also connected to a position sensor 508, which detects the inclination of the detector assembly 500. At some inclination with respect to a vertical axis, the position sensor 508 produces a signal to the microprocessor 502. Based on signals from the position sensor and the movement sensor 506, the microprocessor 502 determines if a patient is likely experiencing a seizure condition.

If a seizure condition is detected, the microprocessor 502 generates an audible alarm from a sound generator 510. In addition to the audible alarm, the microprocessor is also connected to a radio module 512 that is capable of sending radio signals to a base station, which in turn can autodial family members or 911 to provide a notification of the seizure condition.

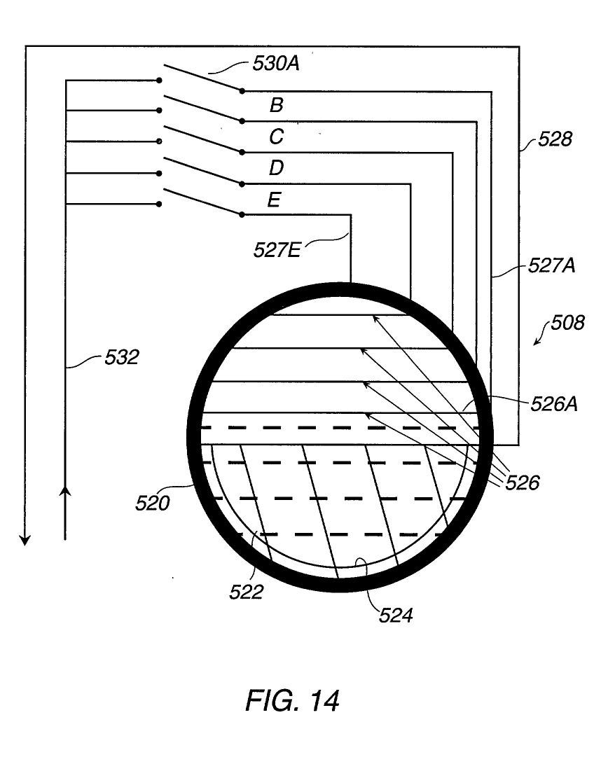

FIG. 14 shows an embodiment of the position sensor 508. In the illustrated embodiment, the position sensor 508 includes a generally spherical shell 520 that is filled with an electrically conductive liquid 522. In one embodiment, the electrically conductive liquid 522 includes mercury. However, other types of liquids can also be employed in other embodiments. The lower portion of the spherical shell 520 is coated with electrically conductive layer 524. The spherical shell 520 is formed of an electrically non-conductive material. In one embodiment, the electrically conductive layer 524 includes electrically conductive paint that is painted onto the inside of the shell 520. Alternatively, the layer 524 can be adhered to the inside of the shell 520. The electrically conductive liquid 522 is in electrical communication with the electrically conductive layer 524.

In addition, electrically conductive lines 526 are also arranged at different levels inside the spherical shell 520. The lines are generally ring-shaped and are coated to the inner wall of the spherical shell 520. Each of the lines 526 is connected to respective one of outside wires 527. Thus, line 526A is connected to wire 527A, line 526B is connected to wire 527B, and so forth. Also, the electrically conductive layer 524 is connected to an electrically conductive line 528.

Switches 530A-E are also arranged along the electrically conductive lines 527A-E. One of the switches 530A-E is closed to enable one of the lines 526A-E. If the switch 530A is closed, then the electrically conductive liquid 522 touching the line 526A would cause a short circuit between line 526A and the electrically conductive layer 524. A relatively shallow inclination from the vertical is needed for the electrically conductive liquid 522 to touch the line 526A. At the other extreme, if the switch 530E is closed, then the electrically conductive liquid 522 will have to touch the line 526E to form a short circuit between the line 526E and the electrically conductive layer 524. This corresponds to a generally horizontal arrangement of the shell 520.

Thus, the switches 530A-E can be set to select how steep an inclination from the vertical is needed to generate an indication that the patient has fallen down or may potentially be in trouble. Instead of this switch any other position switch may be used.

Instructions of the various software routines or modules discussed herein are stored on one or more storage devices in the corresponding systems and loaded for execution on corresponding control units or processors. The control units or processors include microprocessors, microcontrollers, processor modules or subsystems (including one or more microprocessors or microcontrollers), or other control or computing devices. As used here, a “controller” refers to hardware, software, or a combination thereof. A “controller” can refer to a single component or to plural components (whether software or hardware Data and instructions (of the various software routines or modules) are stored in respective storage units, which can be implemented as one or more machine-readable storage media. The storage media include different forms of memory including semiconductor memory devices such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; and optical media such as compact disks (CDs) or digital video disks (DVDs).

The instructions of the software routines or modules are loaded or transported to each device or system in one of many different ways. For example, code segments including instructions stored on floppy disks, CD or DVD media, a hard disk, or transported through a network interface card, modem, or other interface device are loaded into the device or system and executed as corresponding software modules or layers. In the loading or transport process, data signals that are embodied in carrier waves (transmitted over telephone lines, network lines, wireless links, cables, and the like) communicate the code segments, including instructions, to the device or system. Such carrier waves are in the form of electrical, optical, acoustical, electromagnetic, or other types of signals.

While the invention has been disclosed with respect to a limited number of embodiments, those skilled in the art will appreciate numerous modifications and variations there from. It is intended that the appended claims cover such modifications and variations as fall within the true spirit and scope of the invention.

What is claimed is

1. An apparatus for detecting a seizure or other movement disorder of a patient, comprising:
a sensor assembly comprising one or more sound sensors; and
a detector assembly adapted to receive an indication of sound detected by the one or more sound sensors,
the detector assembly adapted to determine if the movement disorder is present based on the indication.

2. The apparatus of claim 1, wherein the detector assembly comprises a computer.

3. The apparatus of claim 1, wherein the detector assembly comprises software executable to perform the determination.

4. The apparatus of claim 1, wherein the sensor assembly further comprises a reference sensor to detect environmental noise.

5. The apparatus of claim 4, wherein the detector assembly is adapted to account for the environmental noise in determining if the movement disorder is present.

6. The apparatus of claim 1, wherein the one or more sound sensors are adapted to sense sound generated by patient movement on a surface.

7. The apparatus of claim 1, wherein the detector assembly is adapted to generate an alarm in response to determining that the movement disorder is present

8. The apparatus of claim 7, wherein the detector assembly is adapted to send an alarm notification to a remote location.

9. The apparatus of claim 1, further comprising a central telemetry system operatively coupled to the sensor assembly.

10. The apparatus of claim 9, wherein the central telemetry system comprises a cardiac telemetry system.

11. The apparatus of claim 9, wherein the detector assembly comprises seizure detection software executable in the cardiac telemetry system

12. The apparatus of claim 1, wherein the sensor assembly is adapted to generate signal waves, and
wherein the detector assembly is adapted to determine if the movement disorder is present based on detecting characteristics of the waves including an angle of a slope of each wave.

13. The apparatus of claim 1, further comprising a video device adapted to continuously receive images of the patient, the video device to save a segment of the received images in response to determining the movement disorder is present.

14. An apparatus for detecting a seizure condition of a patient, comprising:
a sensor assembly comprising one or more sensors selected from the group consisting of a sound sensor, a charge transfer sensor, an accelerometer, a micro-accelerometer, a seismometer, a geophone, a hydrophone, and a fiber-optic sensor,
the sensor assembly adapted to detect patient movement; and
a detector assembly adapted to receive an indication of the patient movement from the sensor assembly and to generate an alarm if the detector assembly determines a seizure condition is present.

15. The apparatus of claim 14, wherein the detector assembly is adapted to send the alarm to a remote location.

16. The apparatus of claim 15, wherein the detector assembly is adapted to receive an acknowledgement of the alarm from the remote location.

17. The apparatus of claim 16, wherein the detector assembly is adapted to contact a sequence of remote locations until a predetermined acknowledgment is received.

18. The apparatus of claim 14, wherein the sensor assembly is adapted to detect patient movement on a surface.

19. The apparatus of claim 14, wherein the sensor assembly is adapted to be worn on the patient.

20. The apparatus of claim 14, further comprising a central telemetry system operatively coupled to the sensor assembly.

21. The apparatus of claim 20, wherein the central telemetry system comprises a cardiac telemetry system.

22. The apparatus of claim 20, wherein the detector assembly comprises seizure detection software executable in the cardiac telemetry system.

23. The apparatus of claim 14, wherein the sensor assembly is adapted to generate signal waves, and
wherein the detector assembly is adapted to determine if the movement disorder is present based on detecting characteristics of the waves including an angle of a slope of each wave.

24. The apparatus of claim 14, further comprising a video device adapted to continuously receive images of the patient, the video device to save a segment of the received images in response to determining the seizure condition is present.

25. An apparatus comprising:
an image sensor adapted to generate images of a patient;
a detector assembly coupled to the image sensor,
the detector assembly comprising an image-processing module adapted to process a series of images to determine if a patient seizure condition is present.

26. The apparatus of claim 25, wherein the image sensor comprises a video camera.

27. The apparatus of claim 25, wherein the detector assembly is adapted to generate an alarm in response to determining the patient seizure condition is present.

28. The apparatus of claim 27, wherein the detector assembly is adapted to send an alarm notification to a remote location.

29. The apparatus of claim 25, further comprising a video recorder adapted to save a segment of received images in response to determining the seizure condition is present.

30. A method of detecting a seizure condition, comprising:
receiving indications of sound from one or more sound detectors, the sound generated by patient movement; and
determining if the seizure condition is present in response to the indications.

31. The method of claim 30, further comprising generating an alarm in response to determining the seizure condition is present.

32. The method of claim 30, further comprising receiving an indication of sound due to environmental noise.

33. The method of claim 32, wherein determining if the alarm is present is based on the indications of sound generated by patient movement and sound due to environmental noise.

34. The method of claim 30, wherein receiving the indications comprises receiving signal waves, and
wherein determining if the seizure condition is present based on characteristics of the waves including an angle of a slope of each signal wave.

35. A method of detecting a seizure condition of a patient, comprising:
receiving data representing one of sound generated by patient movement and video images of the patient; and
determining if the seizure condition is present based on the received data.

36. The method of claim 35, wherein receiving the data comprises receiving the data from one or more sound sensors.

37. The method of claim 35, wherein receiving the data comprises receiving the data from one or more image sensors.

38. A method of detecting a seizure condition of a patient, comprising:
receiving data representative of patient movement from a sensor selected from the group consisting of a sound sensor, a charge transfer sensor, an accelerometer, a micro-accelerometer, a seismometer, a geophone, a hydrophone, and a fiber-optic sensor; and
determining if the seizure condition is present based on the received data.

39. An apparatus for detecting a seizure condition of a patient, comprising:
a movement detector to detect if movement of the patient indicative of a seizure condition is present;
a position detector to detect an inclination of the patient; and
a controller adapted to generate an indication of a seizure condition in response to the movement detector, the position detector or the combined outputs of the movement detector and the position detector.

40. The apparatus of claim 39, wherein the movement detector comprises a device selected from the group consisting of an accelerometer, a micro-accelerometer, a geophone, and a piezoelectric device.

41. The apparatus of claim 39, wherein the position detector comprises a spherical structure containing an electrically conductive fluid.

42. The apparatus of claim 41, wherein the electrically conductive fluid comprises mercury.

43. The apparatus of claim 39, further comprising a communications device adapted to communicate with a remote location to provide an indication of the seizure condition and a location of the patient.

44. The apparatus of claim 43, wherein the communications device is adapted to communicate the alarm condition and the location of the patient using one of cellular network signals, GPS/satellite signals, and two-way pager signals.

45. The apparatus of claim 39, wherein the movement detector is adapted to generate signal waves, and wherein the controller is adapted to generate the indication based on characteristics of the waves including an angle of a slope of each signal wave.

46. A method for detecting a seizure condition of a patient, comprising:
detecting movement of a patient that is indicative of a seizure condition with a movement detector;
detecting an inclination of the patient with a position detector;
receiving signals from the movement detector and the position detector; and
generating an indication of the seizure condition in response to the signals.
